(19) United States
(12) Patent Application Publication
(10) Pub. No.: US 2025/0068925 A1
(43) Pub. Date: Feb. 27, 2025

(54) GENERATIVE INTER-SUBJECT TRANSFER LEARNING APPARATUS AND METHOD THEREOF

(71) Applicant: Korea University Research and Business Foundation, Seoul (KR)

(72) Inventors: Seong Whan LEE, Seoul (KR); Kang YIN, Seoul (KR); Hye Bin SHIN, Gimpo-si (KR); Byeong Hoo LEE, Anyang-si (KR)

(21) Appl. No.: 18/800,792

(22) Filed: Aug. 12, 2024

(30) Foreign Application Priority Data
Aug. 16, 2023 (KR) 10-2023-0106740

(51) Int. Cl. G06N 3/096; A61B 5/372

(52) U.S. Cl. CPC G06N 3/096; A61B 5/372

(57) ABSTRACT

A generative inter-subject transfer learning apparatus includes: a data input unit for receiving source data and target data; a preprocessing unit for removing outliers from the source data and augmenting data; a first encoding unit for generating a feature vector of the source data received from the preprocessing unit and a feature vector of the target data; a generation unit for generating transfer data by reconfiguring the feature vector of the source data, and trained so that a domain of the source data follows a domain of the target data; a second encoding unit for generating a feature vector of the transfer data; and a classification unit for classifying the feature vector of the transfer data generated by the second encoding unit, and trained so that a classification result of the transfer data matches a classification result of the source data.

### CROSS-REFERENCE TO RELATED APPLICATION

The present application claims the benefit of priority to Korean Patent Application No. 10-2023-0106740, filed on Aug. 16, 2023 in the Korean Intellectual Property Office. The aforementioned application is hereby incorporated by reference in its entirety.

### BACKGROUND OF THE INVENTION

### Field of the Invention

The present invention relates to a generative transfer learning apparatus and a method thereof, and more particularly, to a generative transfer learning apparatus and a method thereof, which perform domain adaptation through a novel unsupervised method that emphasizes importance of selection of a space around target data and high-quality source data in the brain-computer interface (BCI) techniques.

### Background of the Related Art

In the brain-computer interface (BCI) techniques, existing techniques have been developed mainly focusing on acquisition and decoding of brain signals through an invasive or non-invasive method (e.g., electroencephalography (EEG)). One of the major problems in this field is that significant difficulties are generated due to inherent deficiencies in the quality and quantity of collected data by the noise of EEG signals and external artifacts. To solve this problem, techniques such as domain generalization (DG) and domain adaptation (DA) may be used to solve the difference in data distribution and distribution shift between subjects. However, since practical implementation of DG is difficult in some cases, DA may be a more practical alternative. Traditional methodologies for DA include parameter correlation hypothesis, subspace learning, and manifold learning, and neural network-based approaches are preferred recently. However, these techniques generally require target domain data attached with a label, and BCI application programs may not use the target domain data in some cases. Therefore, for more comprehensive understanding and review, it is useful to consider an unsupervised domain adaptation (UDA) method that utilizes all signal features. To increase universal applicability of the UDA method, relevant and high-quality source data for effective transfer learning (TL) are required. In particular, since integrity of target data may be damaged by learning the shared distribution between the source domain and the target domain, it is important to maintain integrity of the target data in medical application programs, and therefore it is important to put the quality of source data for transmission in the first place for the sake of optimal results, especially in medical BCI.

### SUMMARY OF THE INVENTION

Therefore, the present invention has been made in view of the problems, and it is an object of the present invention to provide a generative transfer learning apparatus and a method thereof, which can solve major problems within a domain adaptation (DA) paradigm for brain-computer interface (BCI).

Another object of the present invention to provide a generative transfer learning apparatus and a method thereof, which can solve the problem of requiring a large amount of labeled target data that requires a lot of resources and is difficult to obtain.

Another object of the present invention to provide a generative transfer learning apparatus and a method thereof, which have scalability and efficiency so as to be applied to various scenarios.

Another object of the present invention to provide a generative transfer learning apparatus and a method thereof, which have an ability of generalizing transfer data by allowing a model to effectively process various inputs and conditions.

The technical problems to be solved by the present invention are not limited to the technical problems mentioned above, and unmentioned other technical problems can be clearly understood by those skilled in the art from the following description.

To accomplish the objects, according to one aspect of the present invention, there is provided a generative inter-subject transfer learning apparatus comprising: a data input unit 10 for receiving source data and target data; a preprocessing unit 20 for removing outliers from the source data and augmenting data; a first encoding unit for generating a feature vector of the source data received from the preprocessing unit 20 and a feature vector of the target data; a generation unit 40 for generating transfer data by reconfiguring the feature vector of the source data, and trained so that a domain of the source data follows a domain of the target data; a second encoding unit for generating a feature vector of the transfer data; and a classification unit 70 for classifying the feature vector of the transfer data generated by the second encoding unit, and trained so that a classification result of the transfer data matches a classification result of the source data.

In an embodiment of the present invention, the generative inter-subject transfer learning apparatus further comprises a discrimination unit 60 that operates together with the generation unit 40 and the second encoding unit 50 to perform a distribution adaptation training process, wherein the generation unit 40 generates the transfer data from the source data so that a distribution is aligned with the target data, the discrimination unit 60 discriminates the feature vector of the transfer data from the feature vector of the target data, and the generation unit 40 is trained to generate the transfer data until the discrimination unit 60 may not discriminate the feature vector of the target data from the feature vector of the transfer data.

In an embodiment of the present invention, the source data may include an electroencephalogram (EEG) signal, and the generation unit 40 and the classification unit 70 may be trained to perform generalized motor imagery EEG classification on inter-subject EEG signals.

In an embodiment of the present invention, an overall loss function of learning, in which the generative inter-subject transfer learning apparatus performs the generalized motor imagery EEG classification, may include a GAN loss function calculated from the distribution adaptation process between the discrimination unit 60 and the generation unit 40, and a classification loss function calculated from the process in which the classification unit 70 is trained so that the classification result of the transfer data matches the classification result of the source data.

To accomplish the objects, according to another aspect of the present invention, there is provided a generative transfer learning method comprising: a data input step of receiving source data and target data, by a data input unit 10 (S10); a preprocessing step of removing outliers from the source data and augmenting data, by a preprocessing unit 20 (S20); a first encoding step of receiving the augmented data and the target data from the preprocessing unit 20, and generating a feature vector of each of the source data and the target data, by a first encoding unit 30 (S30); a generation step of generating transfer data by reconfiguring the feature vector of the source data, by a generation unit (S40); a second encoding step of generating a feature vector of the transfer data, by a second encoding unit 50 (S50); a discrimination step of training the generation unit 40 so that a domain of the source data follows a domain of the target data as a discrimination unit 60 performs a distribution adaptation process together with the generation unit 40 and the second encoding unit 50 (S60); and a classification step of classifying the transferred feature vector generated by the second encoding unit, by a classification unit 70, and training the classification unit 70 so that a classification result of the transferred feature vector matches a classification result of the feature vector of the source data (S70).

In an embodiment of the present invention, the step of training the generation unit 40 so that a domain of the source data follows a domain of the target data may include the steps of: generating the transfer data from the source data to be aligned with the target data, by the generation unit 40; discriminating the feature vector of the transfer data from the feature vector of the target data, by the discrimination unit 60; and generating the transfer data until the discrimination unit 60 may not discriminate the feature vector of the target data from the feature vector of the transfer data, by the generation unit 40.

In an embodiment of the present invention, the source data may include an electroencephalogram (EEG) signal, and the preprocessing step may include the steps of: removing the outliers using a cbaDBSCAN method; reducing a dimension of the source data using principal component analysis (PCA); and increasing data using MixUp.

In an embodiment of the present invention, the distribution adaptation process may use an inner class transmission mechanism to preserve label information during inter-subject transfer from a source domain to a target domain.

According to an embodiment of the present invention, as data preprocessing for removing outliers and augmenting data and adversarial learning for mapping a source domain to a target domain are included, a generative transfer learning apparatus and a method thereof, which can achieve following effects in the BCI, can be provided.

First, according to an embodiment of the present invention, as a space around target data may well adapt to the changing conditions, rather than sharing a latent space of source and target domains, an end-to-end target transfer framework for providing improved performance can be provided.

Second, according to an embodiment of the present invention, as interpretation of the BCI system is improved by using a small sub-dataset, from which outliers are removed using the cbaDBSCAN method, quality of the original dataset can be improved.

Third, according to an embodiment of the present invention, as a more robust and reliable BCI system that can process a wider range of inputs and conditions is provided by ensuring a more generalized target data distribution, both performance of classification and interpretability of model can be improved.

It should be understood that the effects of the present invention are not limited to the effects described above, but include all effects that can be inferred from the detailed description of the present invention or the configuration of the invention described in the claims.

### BRIEF DESCRIPTION OF THE DRAWINGS

FIG. 1 is a view showing a generative transfer learning apparatus according to an embodiment of the present invention.

FIG. 2 is a flowchart illustrating a generative inter-subject transfer learning method for generalized motor imagery EEG classification according to an embodiment of the present invention.

FIG. 3 is an example of an algorithm showing a framework learning process.

FIG. 4 is a view for explaining the process of removing outliers by the preprocessing unit 20.

FIG. 5 is a view showing classical domain adaptation to preserve label information during inter-subject transfer from a source domain to a target domain.

FIG. 6 is a view showing domain adaptation according to an embodiment of the present invention to preserve label information during inter-subject transfer from a source domain to a target domain.

### DETAILED DESCRIPTION OF THE PREFERRED EMBODIMENT

Since the present invention may make various changes and have various forms, specific embodiments will be illustrated in the drawings and described in detail in the description. However, this is not intended to limit the present invention to a specific disclosed form, and it should be understood to include all changes, equivalents, and substitutes included in the spirit and technical scope of the present invention. While describing each drawing, similar reference numerals are used for similar components.

Unless defined otherwise, all terms used herein, including technical or scientific terms, have the same meaning as commonly understood by those skilled in the art. Terms defined in commonly used dictionaries should be interpreted as having a meaning consistent with the meaning in the context of related technologies, and should not be interpreted in an ideal or excessively formal sense unless explicitly defined in this application.

Hereinafter, preferred embodiments of the present invention will be described in more detail with reference to the accompanying drawings.

A generative transfer learning apparatus according to an embodiment of the present invention may be applied to a brain-computer interface (BCI) system, which is focused on collecting and decoding brain signals using an invasive or non-invasive method such as electroencephalography (EEG), as a generative inter-subject transfer learning apparatus for generalized motor imagery EEG classification. The generative transfer learning apparatus includes data preprocessing for removing outliers and augmenting data and adversarial learning for mapping a source domain to a target domain. Here, the term generalized motor imagery EEG may mean electroencephalogram (EEG) data related to imagined movement of a body part. The term “generalized” means that the BCI system may process these motor imageries. As EEG data of all individuals, as well as specially trained persons, the motor imagery (MI) may mean mental simulation or imagination of movement without actual physical movement. When a person imagines performing a physical action, the brain generates an electrical activity similar to the activity generated when the person actually performs the action. The electrical activity can be detected and measured using the EEG. As an individual is able to control the system using his or her thoughts, the motor imagery may be frequently used in the BCI system. For example, when a person imagines moving the right hand, a command that directs to move to the right is sent to the computer cursor. The difficulty in designing a BCI system is that EEG signals may vary greatly from person to person, even from session to session of the same person. The transfer learning apparatus according to an embodiment of the present invention allows the BCI system to effectively adapt to new individuals and conditions through a domain adaptation (DA) method. That is, the transfer learning apparatus of the present embodiment may improve performance of classifying such imagined movement in EEG signals regardless of where the data comes from.

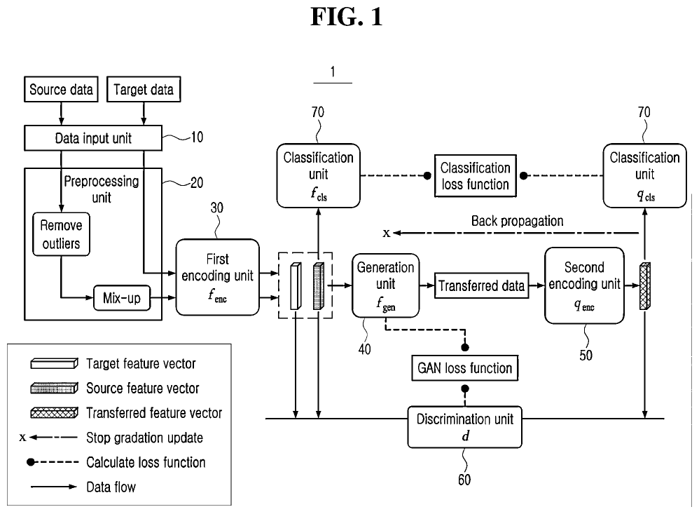

FIG. 1 is a view showing a generative transfer learning apparatus 1 according to an embodiment of the present invention.

Referring to FIG. 1, the generative transfer learning apparatus 1 of the present embodiment may include a data input unit 10, a preprocessing unit 20, a first encoding unit 30, a generation unit 40, a second encoding unit 50, a discrimination unit 60, and a classification unit 70.

The data input unit 10 may receive source data and target data. The data input unit 10 may receive source data (e.g., EEG signals of an original individual group) and target data (e.g., EEG signals of a new individual or group).

The preprocessing unit 20 may perform preprocessing on the input data. The preprocessing unit 20 may remove outliers that can distort the machine learning model from the source data, and augment data through a technique that can improve diversity and representativeness of data without adding noise. Outlier data points (outliers) significantly different from the other data may interfere with performance of the model. The preprocessing unit 20 may identify and remove such outliers by applying a cbaDBSCAN method. In addition, the preprocessing unit 20 may artificially expand the data set using a data augmentation technique such as MixUp. Such a data augmentation process may be applied after removing and before encoding the outliers by a first encoding unit described below. The MixUp may be a process of generating a new training example by taking a convex combination of two training examples. This method may help the model to generalize unseen data better. The process of removing outliers and augmenting data will be described below in more detail.

The first encoding unit may receive the augmented data and target data from the preprocessing unit 20, and generate feature vectors of the source data and target data. The first encoding unit 30 may take and convert the source data and target data, which have been organized and augmented by the preprocessing unit 20, into a low-dimensional representation, which is referred to as a feature vector. That is, the first encoding unit 30 may perform an encoding process to convert raw EEG data into another representation (feature vector) that highlights a function that is most important for the task at hand. The feature vector may represent important characteristics of data that the system should focus on. The purpose of encoding is to extract and represent the most important and relevant features from the data and remove irrelevant detailed information and noise at the same time. Through the conversion process, it is easy to work with the data, and it will be helpful to improve the performance of the steps in latter part of the system.

The generation unit 40 may generate transfer data by reconfiguring the feature vector of the source data. The generation unit 40 may be trained so that the domain of the source data follows the domain of the target data. That is, the generation unit 40 may learn the generation process so that the domain (distribution and characteristics) of the source data follows the domain (distribution and characteristics) of the target data. This is the core of the domain adaptation process of the generative transfer learning apparatus 1, and may be a core process for effectively processing data of a new individual or group.

The second encoding unit 50 generates a feature vector of the transfer data. The second encoding unit 50 may encode the transfer data into a feature vector that the classification unit 70 may use. In this way, as the raw EEG data is converted into a more manageable and useful representation through the encoding process, the system may learn from the data more easily and perform accurate classification.

The discrimination unit 60 may operate together with the generation unit 40 and the second encoding unit 50 to perform a distribution adaptation process. The generation unit 40 may generate transfer data from the source data so that the distribution is more closely aligned with the target data. The discrimination unit 60 may operate to discriminate the transferred feature vector from the feature vector of the target data. The goal is to train the generation unit 40 to generate transfer data until the discrimination unit 60 may not discriminate the feature vector of the target data from the transferred feature vector, while the generation unit 40 generates the transfer data. In this adversarial training process between the discrimination unit 60 and the generation unit 40, a loss function for generative adversarial network (GAN) may be calculated. The overall loss function may include the GAN loss function and a classification loss function, in classification of whether the feature vector of the source data matches the feature vector of the transfer data, which will be described below.

The classification unit 70 may classify the feature vector of the transfer data generated by the second encoding unit. The classification unit 70 may be a part that actually generates a desired system output on the basis of the converted and adjusted input data. The classification unit 70 may be trained in a way that the classification result of the transferred feature vector should match the classification result of the feature vector of the source data. This may be performed to effectively transfer the knowledge obtained from the source data to the target data.

The framework described in FIG. 1 represents a comprehensive approach that includes a series of steps for processing and learning EEG data, and as each of the steps performs an important function for the system to adapt to new data and accurately predict, the major problems in the field of BCI systems, which should be performed in a way of effectively adapting to new data of individuals and maintaining high performance, can be solved.

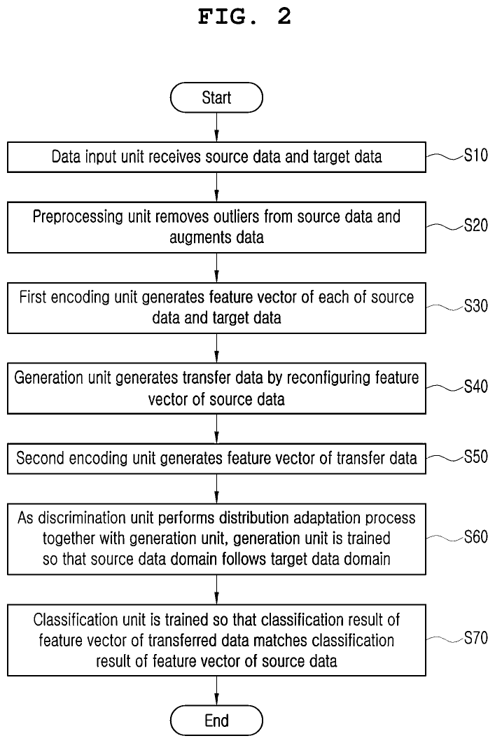

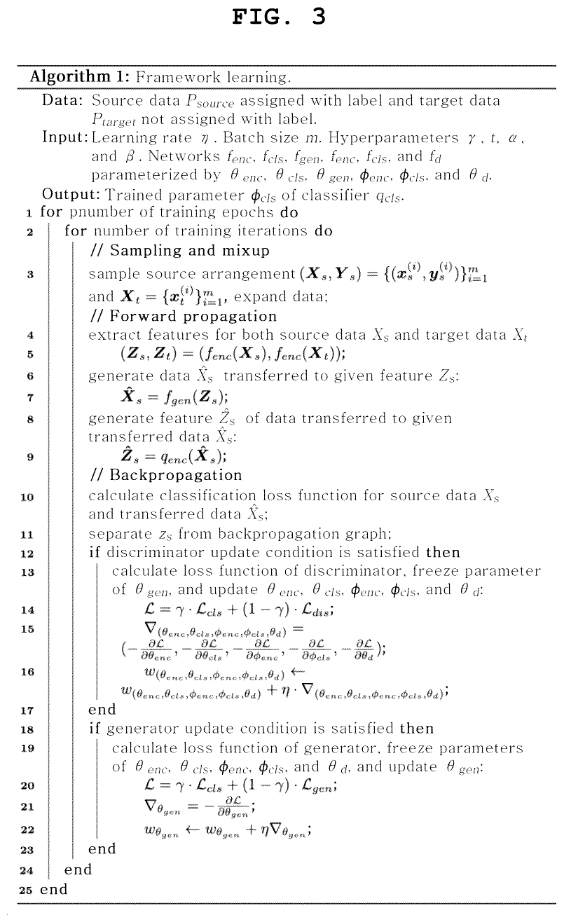

FIG. 2 is a flowchart illustrating a generative inter-subject transfer learning method for generalized motor imagery EEG classification according to an embodiment of the present invention. FIG. 3 is an example of an algorithm showing a framework learning process.

Referring to FIGS. 2 and 3, in a generative inter-subject transfer learning method for generalized motor imagery EEG classification according to an embodiment of the present invention, first, the data input unit 10 receives source data and target data (S10). The preprocessing unit 20 removes outliers from the source data and augments data (S20). The first encoding unit 30 receives the augmented data and the target data from the preprocessing unit 20 and generates a feature vector of each of the source data and the target data (S30). The generation unit 40 generates transfer data by reconfiguring the feature vector of the source data (S40). The second encoding unit 50 generates the feature vector of the transfer data (S50). The discrimination unit 60 operates together with the generation unit 40 and the second encoding unit 50 to perform a distribution adaptation process (S60). According thereto, the generation unit 40 may be trained so that the domain of the source data follows the domain of the target data. The classification unit 70 classifies the transferred feature vector generated by the second encoding unit, and may be trained in a way that the classification result of the transferred feature vector should match the classification result of the feature vector of the source data (S70).

In a framework provided by the generative inter-subject transfer learning method for generalized motor imagery EEG classification described above, (Xₛ,Yₛ)={(xₛ⁽ⁱ⁾,yₛ⁽ⁱ⁾)}ᵢ₌₁^{m} and xₜ={(xₜ⁽ⁱ⁾)}ᵢ₌₁^{m} are data randomly sampled from source domain p and target domain q, respectively, where m»n, where n is the number of total data samples. According to the Bayesian theory, q(Y|X)=q(X|Y)q(Y)/q(X). In the embodiment of the present invention, the goal is to learn a model that maximizes the posterior probability q(Y|X), i.e., q(X|Y)/q(Y). Traditional methods assume that p(X|Y)=q(X|Y) by themselves or learn a shared distribution for the data sampled from p and q. On the contrary, in the present embodiment, a shared label space is assumed for two domains, and it is attempted to learn the mapping ƒ_{θ}:Xₛ→Xₜ.

$$arg {max}_{∅} q ( y \vert {X}_{t} , ∅ ) = arg {max}_{∅ , θ} q ( Y \vert ( {X}_{s} , θ ) ; ∅ ) = arg {max}_{∅ , θ} q ( f ( {X}_{s} , θ ) \vert Y , ∅ ) q ( Y )$$

Here, ƒ(Xₛ,θ) is conversion from the source domain to the target domain, which can be parameterized by θ. Function q(Y|Xₜ,∅) is a prediction model in the target domain, which can be parameterized by ϕ. In the present embodiment, a method of converting a source domain to a target domain can be found using the matching, and at the same time, the posterior probability of transfer data can be maximized by jointly optimizing θ and ϕ.

The mapping network ƒ_{θ} described above is implemented as an encoder-generator structure f_{enc} and f_{gen} as shown in FIG. 1, and the prediction model q_{∅} may be implemented by another encoder q_{enc} and q_{cls}. The source data may be first encoded into a high-dimensional vector by f_{enc} and then reconfigured by f_{gen}. The mapping process is trained in an adversarial manner, and the discrimination unit 60 may be used to evaluate whether the transfer data belongs to the target domain.

In addition, to guarantee a high-quality training data group as described above, the process of removing outliers and augmenting data may be performed by the preprocessing unit 20 before the data is supplied to ƒ_{θ}. The entire framework is trained in an end-to-end manner, and q_{enc} and q_{cls} may be prepared at the testing step immediately after the training.

Hereinafter, after further describing the process of removing outliers and augmenting data, an inter-subject transfer module will be described.

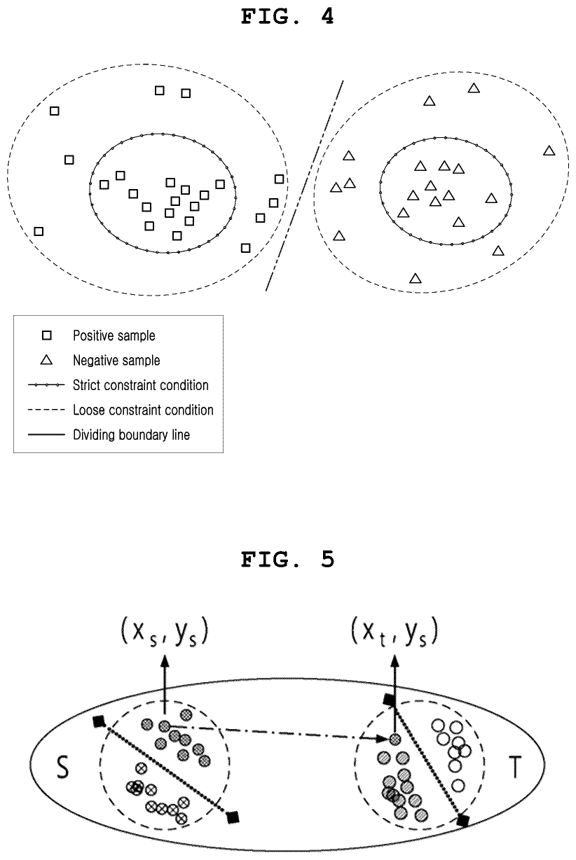

FIG. 4 is a view for explaining the process of removing outliers by the preprocessing unit 20.

Referring to FIG. 4, as described above, the preprocessing unit 20 may remove outliers from the source data and augment data (S20). At the step of collecting EEG signals, quality of collected data may be degraded due to various factors such as environmental disturbances, distraction or drowsiness of the subject, and the like. When the outliers are included in the training data, performance may be lowered significantly.

FIG. 4 shows an example of binary classification, and the adaptive model learns a linearly separable boundary in the latent space using positive and negative training samples of the source domain. In addition, in a real-time BCI, conventional methods ignore selection of adaptive samples since training using a large amount of data takes a long time and is burdensome to participants, but in the present embodiment, using all training samples (the inner side of a circle with a dotted outer line) is avoided, and a subset of high-quality samples (inner circle) is selected instead. Samples inside the inner circle include a more essential and substantive class-related pattern. Other samples may be more vulnerable to noise. Therefore, outlier removal may be performed before training the model on the basis of two assumptions described below.

First, distribution of a latent space may be obtained directly without training.

Second, subsets of high-quality data belonging to different classes are independent

and equally distributed.

A fixed data distribution may be obtained through the first assumption, and the second assumption supports the need of outlier removal. Essentially, this process is configured of two steps of dimension reduction and clustering. At the first step, the dimension of source data is reduced using a principal component analysis (PCA).

$$A = H \overline{{X}_{s}} V$$

Here, X̅ₛ̅ is the mean of channels of Xₛ, and H and V are the centralized matrix and the eigenvector matrix, respectively. Data after dimension reduction may be expressed as A.

Density-based spatial clustering (DBSCAN) of application programs with noise is suitable for finding clusters of an arbitrary shape, and this is suitable for finding outliers in the case of the present embodiment. However, this method also has difficulties in dealing with various densities and several limitations such as sensitivity to parameters, and the like.

In order to maintain the balance of the number of samples in each class after removing the outliers, class-balanced automatic adaptive DBSCAN (cbaDBSCAN) based on the original one is applied in the present embodiment. In particular, clustering is performed within each class. That is, in the case of a k-category classification task having m training samples, the data is divided into k groups as shown in the following equation.

$$A = {⋃}_{j = 1}^{k} {A}_{j}$$

Here, each group may include only samples of a single class as shown below in the equation.

$${A}_{j} = {\{ {x}_{s}^{( i )} \vert {y}_{s}^{( i )} = j \}}_{i = 1}^{m}$$

DBSCAN is used for each group.

$${\ddot{A}}_{j} = DBSCAN ( {A}_{j} ; ϵ , min Pts )$$

Here, Äⱼ represents the minimum neighbor minPts, which sets samples in the reserved cluster determined by the hyperparameter radius ϵ and the threshold value for the outlier tolerance. Since imbalance of samples occurs for each class and subject when a common e is set for different groups, individual Äⱼ is calculated for each group.

Once the samples reserved for all k categories are obtained like Ä=Uⱼ₌₁^{k}Äⱼ, the samples are mapped. The index is returned to the raw data Xₛ to obtain selected \ddot{X}ₛ. For simplicity, the overhead is omitted. The data after removing the outliers are represented as Xₛ using an accent notation.

Meanwhile, the data augmentation (mixup) method performed by the preprocessing unit 20 mitigates the limitation due to the removal of outliers by increasing the amount of training data. Since the target data label may not be used at this step, the mixup may be applied only to the source data. In particular, m training sample groups (Xₛ,Yₛ)={(xₛ⁽ⁱ⁾,yₛ⁽ⁱ⁾)}ᵢ₌₁^{m} of source data are considered. In the present embodiment, γ₁˜β(α,α) may be randomly sampled in the following method.

$${x}_{s}^{( i )} ← {λ}_{1} \cdot {x}_{s}^{( i )} + ( 1 - {λ}_{1} ) \cdot {x}_{s}^{( j )}$$

$${y}_{s}^{( i )} ← {λ}_{1} \cdot {y}_{s}^{( i )} + ( 1 - {λ}_{1} ) \cdot {y}_{s}^{( j )}$$

Here, i represents the index of the training sample in a range of 1, 2, . . . , m or more, and j is a randomly selected index (j≠i).

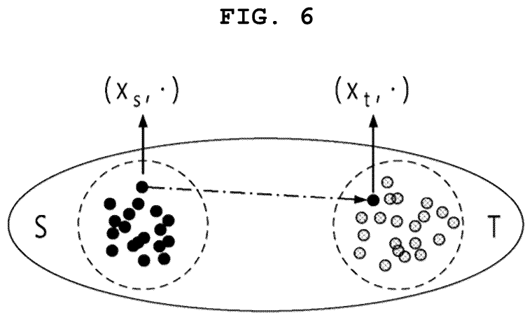

FIG. 5 is a view showing classical domain adaptation to preserve label information during inter-subject transfer from a source domain to a target domain. FIG. 6 is a view showing domain adaptation according to an embodiment of the present invention to preserve label information during inter-subject transfer from a source domain to a target domain.

As described above, the discrimination unit 60 operates together with the generation unit 40 and the second encoding unit 50 to perform a distribution adaptation process (S60). According thereto, the generation unit 40 may be trained so that the domain of the source data follows the domain of the target data. The classification unit 70 classifies the transferred feature vector generated by the second encoding unit, and may be trained in a way that the classification result of the transferred feature vector should match the classification result of the feature vector of the source data (S70).

Referring to FIGS. 5 and 6, in the present embodiment, adversarial learning is utilized to identify and mitigate domain mismatch to solve the gap between the source domain and the target domain as a domain adaptation strategy. The discrimination unit 60 is designed to discriminate source data representation from target data representation in the latent space, while the generation unit 40 attempts to transmit the characteristic style of the source data to the target domain in order to deceive the discrimination unit 60 to make a wrong classification.

Initially, source data Xₛ and target data Xₜ may be processed by f_{enc} to obtain latent space representation Zₛ and Zₜ, respectively. Then, the Zₛ representation may be reconfigured by f_{gen} to be a signal \hat{X}ₛ of the same size in the raw data space. Thereafter, transfer data \hat{X}ₛ is supplied to auxiliary q_{enc} to obtain corresponding embedding \hat{Z}ₛ.

In the GAN training, the generation unit 40 and the discrimination unit 60 may be updated alternately. To train the discrimination unit 60, an input configured of a pair of a source domain and a target domain may be used as true and false samples, respectively. In particular, m pairs of data may be randomly sampled, and loss of the discrimination unit 60 may be calculated using the following formula, in which d is the discriminator which is the subject of discrimination unit 60.

$${L}_{d i s} = \frac{1}{m} [ {log}_{d} ( {z}_{s}^{( i )} ) + log ( 1 - d ( {z}_{s}^{( i )} ) ) + {log}_{d} ( {\hat{z}}_{s}^{( i )} ) ]$$

To mitigate the effect of specificity of individual samples and emphasize dominant characteristics of the target domain, a mean of the target samples is calculated to obtain the center point of each arrangement as shown below in the equation.

$${\overline{z}}_{t} = \frac{1}{m} {Σ}_{i = 1}^{m} {z}_{t}^{( i )}$$

In addition, loss of the discrimination unit 60 may be updated accordingly by the equation shown below.

$${L}_{d i s} = \frac{1}{m} {Σ}_{i = 1}^{m} [ {log}_{d} ( {z}_{s}^{( i )} ) + {log}_{d} ( {\hat{z}}_{s}^{( i )} ) ] + log ( 1 - d ( {\overline{z}}_{t} ) )$$

During the training of the generation unit 40, only the transmitted samples, not the paired samples, are focused on. As a result, loss of the generation unit 40 may be formulated as shown below.

$${L}_{d i s} = \frac{1}{m} {Σ}_{i = 1}^{m} log ( 1 - d ( {\hat{z}}_{s}^{( i )} ) )$$

Meanwhile, an “inner class transmission” mechanism is introduced to preserve label information during inter-subject transfer from a source domain to a target domain. As described in FIGS. 5 and 6, this mechanism maintains consistency of labels during transmission by utilizing label information that can be used in the source domain. Accordingly, the transfer data maintains both domain and class knowledge.

To realize this mechanism, the label of the transfer data may be directly assigned to match the label of corresponding source data. The reason is that it is desired to transmit only the information related to the domain without changing the label information. In the present embodiment, the label of the source data is directly taken as a pseudo label and assigned to the transfer data, and this is actually simpler and conceptually reasonable. Therefore, q_{cls} is trained in the target domain while utilizing f_{cls} at the same time, and the classification loss is defined as shown below.

$${L}_{d i s} = \frac{1}{m} {Σ}_{i = 1}^{m} {y}_{s}^{( i )} [ {log}_{{f}_{c l s}} ( {z}_{s}^{( i )} ) + {log}_{{q}_{c l s}} ( {\hat{z}}_{s}^{( i )} ) ]$$

The classification loss may be used in the update step of both the discrimination unit 60 and the generation unit 40. Therefore, the overall loss function may be formulated as follows.

$$ℒ = {γℒ}_{c l s} + {ℒ}_{g e n} / {ℒ}_{d i s}$$

Here, γ functions as a hyperparameter for balancing the classification loss and the GAN loss. L_{gen} and L_{dis} are not used simultaneously.

According to an embodiment of the present invention, an end-to-end inter-subject transmission framework that provides improved performance can be provided by learning a space based on target data using a non-abnormal small subset.

According to an embodiment of the present invention, as outliers are removed by the cbaDBSCAN method, quality of an original dataset can be improved by improving interpretation of the BCI system by utilizing a smaller sub-dataset without outliers.

According to an embodiment of the present invention, as distribution of learned target data is further generalized and leads to a more powerful and reliable BCI system that can handle a wide range of inputs and conditions, both classification performance and model interpretability can be improved.

According to an embodiment of the present invention, the apparatus and method may be utilized for the general public, as well as patients having difficulties in moving the body (paralyzed patients, stroked patients, and the like) and disabled people, and the technique may also be applied to smart home control or artificial intelligence techniques based on a brain-computer interface. In addition, the present embodiment may be expanded to artificial intelligence techniques, and for example, may be utilized for controlling robots or the like moving only by thoughts of a user.

The description of the present invention described above is for illustrative purposes, and those skilled in the art will understand that the present invention may be easily modified in other specific forms without changing the technical spirit or essential features of the present invention. Therefore, the embodiments described above should be understood in all respects as illustrative and not restrictive. For example, each component described as a single form may be implemented in a distributed manner, and similarly, components described as distributed may also be implemented in a combined form.

The scope of the present invention is indicated by the patent claims described below, and all changes or modified forms derived from the meaning and scope of the claims and their equivalent concepts should be construed as being included in the scope of the present invention.

### DESCRIPTION OF SYMBOLS

1: Generative transfer learning apparatus
10: Data input unit
20: Preprocessing unit
30: First encoding unit
40: Generation unit
50: Second encoding unit
60: Discrimination unit
70: Classification unit

1-8. (canceled)

9. A generative inter-subject transfer learning apparatus, comprising:
a data input unit configured to receive source data and target data;
a preprocessing unit configured to remove outliers from the source data and augment the source data to obtain augmented data;
a first encoding unit configured to generate
a feature vector of the source data, based on the augmented data received from the preprocessing unit, and
a feature vector of the target data;
a generation unit configured to perform a generation process for generating transfer data by reconfiguring the feature vector of the source data, wherein the generation unit is trained to perform the generation process so that a domain of the source data follows a domain of the target data;
a second encoding unit configured to generate a feature vector of the transfer data; and
a classification unit configured to classify the feature vector of the transfer data generated by the second encoding unit, wherein the classification unit is trained so that a classification result of the feature vector of the transfer data matches a classification result of the feature vector of the source data.

10. The apparatus according to claim 9, further comprising:
a discrimination unit configured to operate together with the generation unit and the second encoding unit to perform a distribution adaptation training process, in which
the generation unit generates the transfer data from the source data so that a distribution of the source data is aligned with a distribution of the target data,
the discrimination unit discriminates the feature vector of the transfer data from the feature vector of the target data, and
the generation unit is trained to generate the transfer data until the discrimination unit no longer discriminates the feature vector of the target data from the feature vector of the transfer data.

11. The apparatus according to claim 10, wherein the source data include an electroencephalogram (EEG) signal, and the generation unit and the classification unit are trained to perform generalized motor imagery EEG classification on inter-subject EEG signals.

12. The apparatus according to claim 11, wherein
an overall loss function of learning, in which the generative inter-subject transfer learning apparatus performs the generalized motor imagery EEG classification, includes:
a GAN loss function calculated from a distribution adaptation process between the discrimination unit and the generation unit, and
a classification loss function calculated from a process in which the classification unit is trained so that the classification result of the feature vector of the transfer data matches the classification result of the feature vector of the source data.

13. A generative transfer learning method comprising:
a data input step of receiving source data and target data, by a data input unit;
a preprocessing step, by a preprocessing unit, of removing outliers from the source data and augmenting the source data to obtain augmented data;
a first encoding step, by a first encoding unit, of
generating a feature vector of the source data, based on the augmented data received from the preprocessing unit, and
generating a feature vector of the target data;
a generation step, by a generation unit, of perform a generation process for generating transfer data by reconfiguring the feature vector of the source data;
a second encoding step, by a second encoding unit, of generating a feature vector of the transfer data;
a discrimination step of training the generation unit to perform the generation process so that a domain of the source data follows a domain of the target data, wherein, in the discrimination step, a discrimination unit performs a distribution adaptation process together with the generation unit and the second encoding unit; and
a classification step, by a classification unit, of classifying the feature vector of the transfer data generated by the second encoding unit, and training the classification unit so that a classification result of the feature vector of the transfer data matches a classification result of the feature vector of the source data.

14. The method according to claim 13, wherein the discrimination step of training the generation unit includes the steps of:
generating, by the generation unit, the transfer data from the source data to be aligned with the target data;
discriminating, by the discrimination unit, the feature vector of the transfer data from the feature vector of the target data; and
generating, by the generation unit, the transfer data until the discrimination unit no longer discriminates the feature vector of the target data from the feature vector of the transfer data.

15. The method according to claim 14, wherein the source data include an electroencephalogram (EEG) signal, and the preprocessing step includes the steps of:
removing the outliers using a cbaDBSCAN method;
reducing a dimension of the source data using principal component analysis (PCA); and
increasing data using MixUp.

16. The method according to claim 15, wherein the distribution adaptation process uses an inner class transmission mechanism to preserve label information during inter-subject transfer from a source domain to a target domain.
